(19) United States
(12) Patent Application Publication
(10) Pub. No.: US 2015/0232907 A1
(43) Pub. Date: Aug. 20, 2015

(54) Automatic Apparatus for Risk Assessment of Chemicals

(71) Applicant: Eutomation, Inc., Fremont, CA (US)

(72) Inventors: Xuejun Wang, Pleasanton, CA (US); Xinhua Zong, San Jose, CA (US); Michael Z. Wong, Castro Valley, CA (US); Kai-An Wang, Cupertino, CA (US)

(21) Appl. No.: 14/604,980

(22) Filed: Jan. 26, 2015

Related U.S. Application Data
(60) Provisional application No. 61/941,379, filed on Feb. 18, 2014.

(51) Int. Cl. C12Q 1/02; G01N 15/14; G01N 35/00; G01N 35/10; G01N 35/02

(52) U.S. Cl. CPC C12Q 1/025; G01N 35/1065; G01N 35/1011; G01N 35/025; G01N 35/0099; G01N 15/1463; G01N 2035/0444

(57) ABSTRACT

An automated toxicity assessment apparatus includes multiple sample plates each configured to hold test wells for holding a test solution comprising a test compound and a test organism. A dispense head holds dispense tips for dispensing test solutions into the test wells in one sample plate at each time. An electronic detector can capture an image of a test solution in one of test wells on a sample plate to establish a dose-response curve for the test organism. A carousal system rotates a first sample plate to a first position under the dispense head to allow the plurality of dispense tips to dispense solutions into the test wells on the first sample plate. The carousal system rotates the first sample plate to a second position to allow the electronic detector to capture an image of a test solution in the one of test wells on the first sample plate.

### BACKGROUND OF THE INVENTION

The present invention relates to an automatic apparatus and methods for evaluation of toxicities of test compounds in a high throughput manner.

Chemicals are used to make virtually every man-made product. The increase and widespread use of chemicals pose great risks to human health and the environment. Chemical hazard refers to the inherent properties of a substance that causes harm to human health or the environment. Risk is the possibility of a harmful event arising from specific uses and circumstances of exposure to a substance. Toxicity assessment is a major component of risk assessment. Toxicity assessment provides an estimate on how much and what kind of harm that a substance causes. In today's society, it is important to assess the toxicity of both known compounds and especially new compounds not found in nature. Before a compound or a composition is commercialized, the toxicity of the compound or compounds contained in the composition must be tested.

Toxic effects are classified as either acute or chronic. Acute toxic effects are estimated by LD50 (Lethal Dose for 50% of the animals) studies or observations of accidental exposures. LD50 determines the amount of a substance that kills half the test animals after a specified exposure time. Traditional methods of assessing the mean lethal dose LD50 use death of animals as an endpoint. Another approach is to observe clear signs of toxicity at one of a series of fixed dosage levels often referred to as Fixed Dose Procedures. Toxicity evaluations based on the death of animals as an endpoint are increasingly under public and institutional scrutiny and are being phased out in favor of tests such as fixed dose procedures. The U.S. Food and Drug Administration has begun to approve non-animal alternatives in response to research cruelty concerns and the lack of validity of animal tests as they relate to humans. In vitro testing methods have been developed to replace animals from the toxicity tests. In vitro procedures comprise testing effects of chemical compounds on cultured bacterial or mammalian cells. When in vitro procedures are used, a dose-response relationship is often determined and endpoints such as No Observable Effect Level (NOEL) and Inhibition Concentration 50% (IC50) are established. Although consuming less funds compared to methods comprising the use of animals, the culture of microbial and cell cultures is both quite time consuming and costly.

To speed up chemical safety assessments and replace outdated animal testing methods, there is clearly a need for high-throughput toxicity measuring technologies.

### SUMMARY OF THE INVENTION

The present application pertains to automated apparatus and methods for high-throughput screening of toxicity of chemicals. The presently disclosed automated apparatus and methods can include one or more of the following advantages. The disclosed apparatus and methods can conduct higher throughput screening of toxicity of test compounds than conventional systems. The present disclosed automated apparatus and methods can prevent operator errors such as sample misplacement and cross contamination. The test compound can be automatically diluted to establish a wide range of toxicant concentrations in a continuous high throughput format, with minimal operator intervention.

The disclosed apparatus provides an efficient way to generate dose-response data which provide information on the levels of an unknown toxicant. The disclosed apparatus can enable automatic replacement and cleaning of test wells, which allows easy replication of tests to improve the statistical interpretation of the results. The disclosed apparatus can thus reduce the time and cost spent for assessing toxicities of the test compounds.

In one general aspect, the present invention relates to an automated toxicity assessment apparatus which includes: a plurality of sample plates each configured to hold multiple test wells that each can hold a test solution comprising a test compound and a test organism, wherein the plurality of sample plates comprises a first sample plate; a dispense head that can hold a plurality of dispense tips that can dispense test solutions into the test wells in one of the plurality of sample plates at each time; an electronic detector that can capture an image of a test solution in one of test wells in one of the plurality of sample plates, which allows a dose-response curve to be established for the test organism; and a carousal system that can rotate the first sample plate to a first position under the dispense head to allow the plurality of dispense tips to dispense solutions respectively into the test wells on the first sample plate, wherein the carousal system can rotate the first sample plate to a second position to allow the electronic detector to capture an image of a test solution in the one of test wells on the first sample plate.

Implementations of the system may include one or more of the following. The dispense head can hold a plurality of probes that can measure oxygen levels, pH values, or conductivities in the test solutions in the test wells on the first sample plate. The automated toxicity assessment apparatus can further include a robotic arm that can lower the dispense head to engage the plurality of dispense tips respectively with the test wells on the first sample plate when the first sample plate is rotated to the first position by the carousal system. The robotic arm can lower the dispense head to engage the plurality of probes respectively with the test wells on the first sample plate when the first sample plate is rotated to the first position by the carousal system. The automated toxicity assessment apparatus can further include a rotational mechanism that can rotate the first sample plate when the first sample plate is moved to the second position the carousal system, thereby allowing different test wells to be moved to the vicinities of the electronic detector and images of the different associated test solutions to be captured by the electronic detector. The automated toxicity assessment apparatus can further include a liquid transfer mechanism that can transfer the test solution to the plurality of dispense tips. The liquid transfer mechanism can include a syringe that can draw the test solution from a storage unit and pump the test solution towards the plurality of dispense tips. The automated toxicity assessment apparatus can further include a drain channel in the first sample plate and configured to drain test solutions from the test cells. The automated toxicity assessment apparatus can further include a filter in the first sample plate and configured to filter the test solutions to be drained from the test cells. The test organism can include green alga selenastrum capricornutum.

These and other aspects, their implementations and other features are described in details in the drawings, the description and the claims.

### BRIEF DESCRIPTION OF THE DRAWINGS

FIG. 1 is a perspective view of an automated toxicity measuring apparatus in accordance with the present invention.

FIG. 2 is a top view of the automated toxicity measuring apparatus showing a carousel system, sample holders, an electronic detector, and a robotic arm, a dispense head, and an electronic control unit.

FIG. 3 shows a front view of the automated toxicity measurement apparatus showing an electronic detector, a robotic arm, drain channels, probes and a dispense tip.

FIG. 4A shows the dispense tip and the probe for adding testing solution and monitoring test conditions.

FIG. 4B is a detailed view showing the drain channels for replacing used testing toxicant solution.

### DETAILED DESCRIPTION OF THE INVENTION

In the present disclosure, the chemical compound of which the toxicity is assessed is hereinafter referred to as test compound. Inherent toxicity to non-human organisms is evaluated based on aquatic toxicity data. Examples of chemical compounds suitable for toxicity testing by the presently disclosed apparatus include naturally occurring but not widely used commercially compounds, and new compounds such as non-naturally-occurring new chemical entities. The latter are often synthesized by the chemical industry such as pharmaceutical and agrochemical companies. The presently disclosed apparatus and method are also suitable for testing toxicities of other chemicals.

Toxicity herein refers to a relationship between the dose of a compound and its effect on an exposed organism. For additional clarity, the organism of which is exposed to test compound is hereafter referred to as test organisms. In some embodiments, exemplified test organisms include green alga selenastrum capricornutum. A test organism is exposed in a static system to a series of concentrations of effluent, or to receiving water, for a predetermined time period as specified by standard USEPA or ASTM methods. The response of the population is measured in terms of changes in cell density (cell counts per mL), biomass, chlorophyll content, or absorbance.

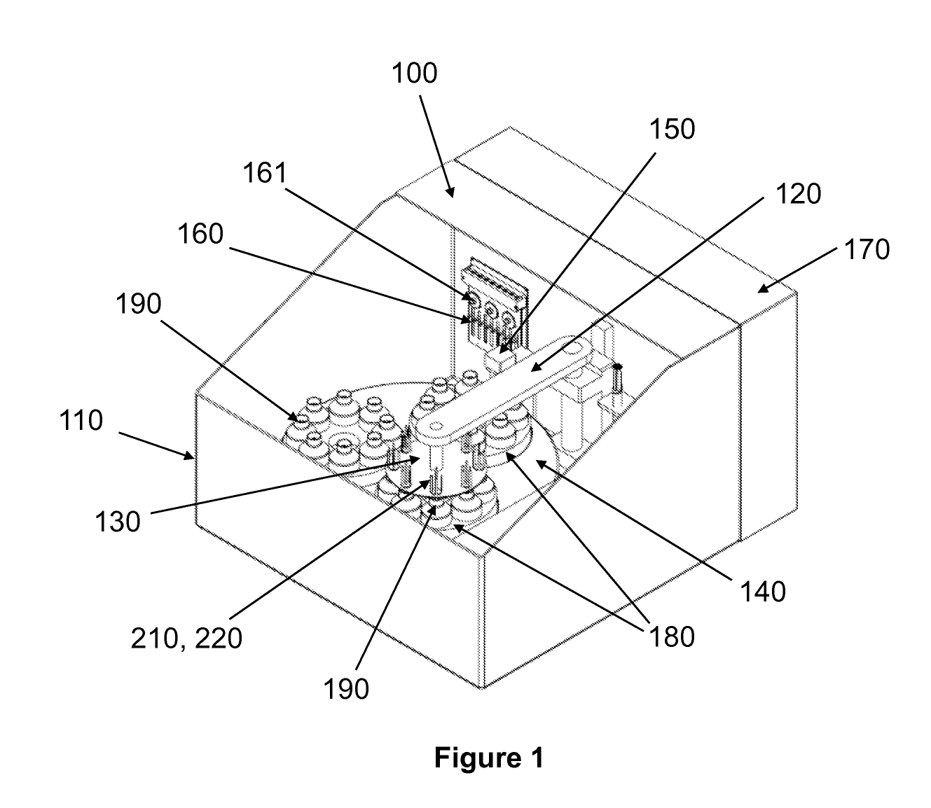

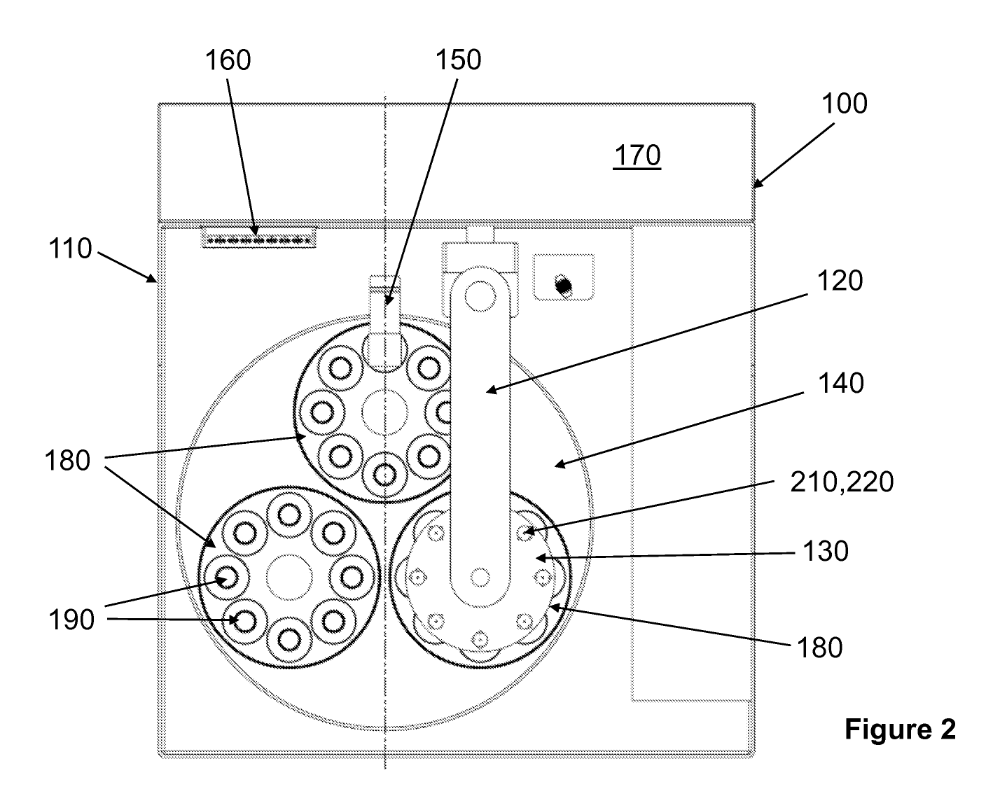

Referring to FIGS. 1 and 2, an automated toxicity assessment apparatus 100 includes an enclosure 110, a robotic arm 120, a dispense head 130 mounted with dispense tips 210 and multiple probes 220, a carousel system 140, an electronic detector 150, a liquid transfer syringe 160, an electronic control unit 170. The enclosure 110 can be an environmental controlled chamber or incubator, which can provide “cool-white” fluorescent illumination, temperature control (i.e. 25±1° C.), or atmosphere conditions (humidity, oxygen/carbon dioxide level) optimal to the growth of test organisms. The carousel system 140 can rotate multiple sample holders 180 that each holds multiple test well 190 serving as test chambers configured to hold aqueous test solutions comprising test compounds and test organisms. The carousal system 140 can position test wells 190 to the vicinities underneath dispense head 130. The robotic arm 120 can lower the dispense head 130 to the vicinities above the respective individual test wells 190. The dispense tips 210 are fluidically connected to the liquid transfer syringes 160 via respective liquid transfer channels, which transfer accurate amounts of test samples and reagents into the test wells 190. The syringe 160 is fluidically connected to multi-port valves 161 with a selective means for taking aqueous stock solutions from different storage units. The probes 220 can monitor and record testing conditions (i.e. oxygen, pH or conductivity levels) of liquids inside of test well 190. The electronic detector 150 collects a digital image of the solution comprising of test organisms and test compound after a lapse of a predetermined time period from the mixing of test organisms and test compound solution. The electronic control unit 170 provides electrical or mechanical control or the automated operations of the above described components.

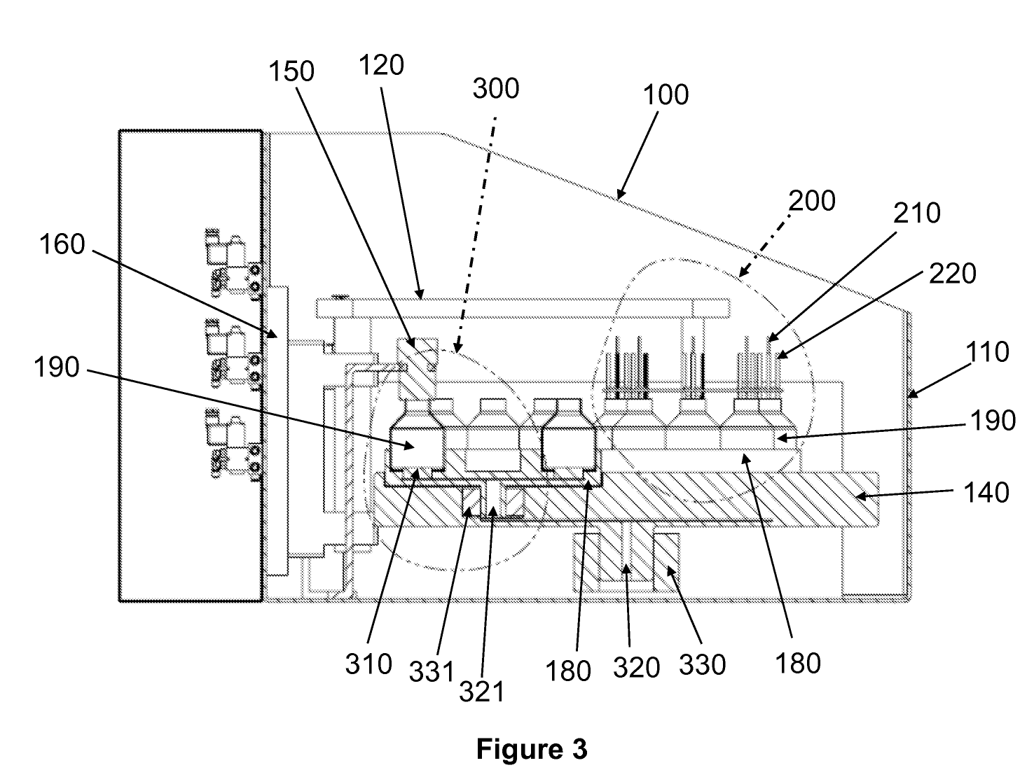

In some embodiments, the toxicity level in a sample solution in a test well 190 is evaluated by measuring the cell density (cells/mL) of test organisms as a function of the concentration of test compound. Based on the dose response of the cell density of test organisms, data correlating to the toxicity of test compound is obtained. Solutions comprising the test compound are introduced into multiple test wells 190 which are preloaded with fixed amount of test organisms. Referring now to FIG. 3, the sample holders 180 can be rotated by the carousel system 140 to different positions to receive liquid dispense or for taking measurement. The dispense head 130 mounted with dispense tips 210 and multiple probes 220 can be moved up and down by the robotic arm 120. The down position (as shown in FIG. 3) of the robotic arm 120 allows liquids to be dispensed into the test well 190 and for the measurement of oxygen/pH/conductivity levels of the solutions inside the test well 190. In the up position, the dispense tip 210 and the probes 220 are disengaged so that the next carousal 180 can be moved under the dispense head 130 for liquid dispense.

Each sample holder 180 can be rotated by a rotational mechanism 330, which can be implemented by a pulley and belt mechanism driven by a stepping motor (not shown). The rotational mechanism 330 can position test wells 190 under the dispense tips 210 and the probes 220 mounted on the dispense head 130. At each rotational stop of the rotational mechanism 330, liquids can be dispensed in parallel from multiple channels into multiple test cells 190 mounted a same sample plate 180.

The rotational mechanism 330 can also rotate the test wells 190 to be under the electronic detector 150. As described above, the dispense tips 210 are fluidically connected to the syringe 160 which draws a culture nutrient solution from a storage unit. The dispense tips 210 can discharge the stock solution into each test well 190 under the control of a piston operation mechanism (not shown) that controls the amount of nutrient solution discharged, and a syringe transportation mechanism (not shown). The piston operation mechanism can include a stepping motor and ball screw, a pulley and belt to control the movement of piston through the operation of the stepping motor. The syringe 160 can be adjusted in up/down directions by for example a pulley-and-belt mechanism, ball spline and a stepping motor. Thus the movement of the syringe 160 can be driven by the stepping motor, and the amount of the injected solution can be quantitatively controlled.

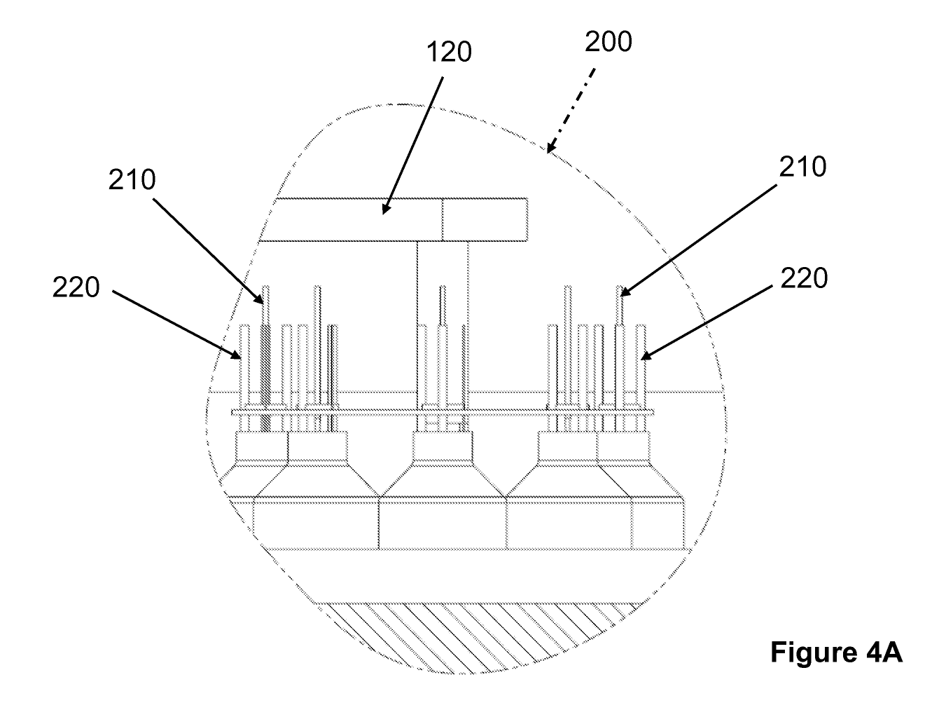

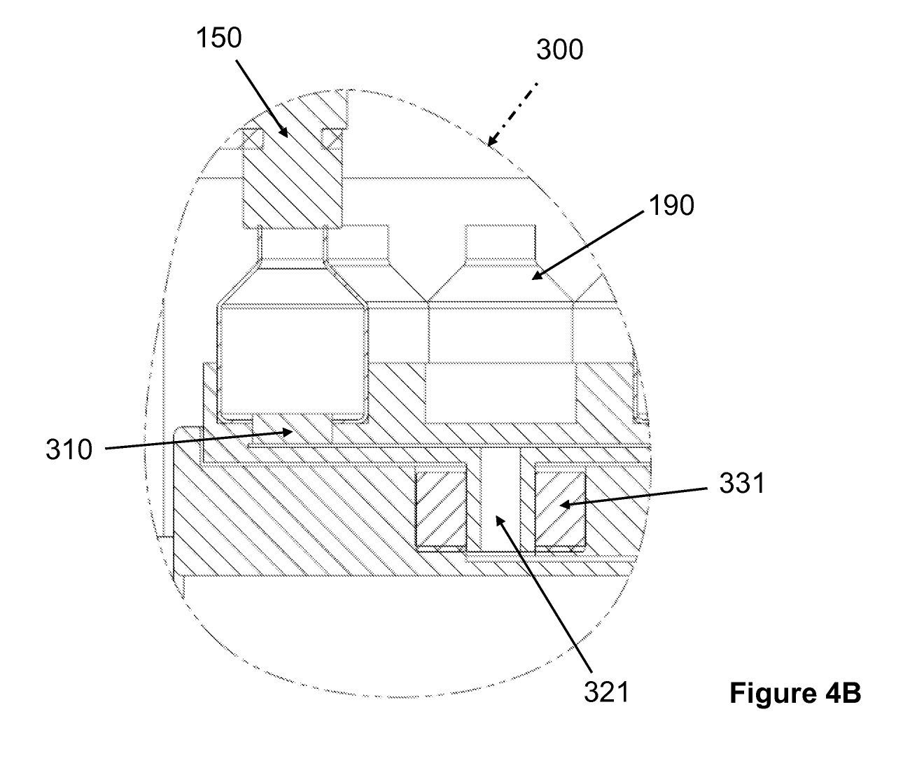

Referring to FIGS. 3 and 4A (enlarge portion 200), the test solutions in the test cells 190 are formed by serial dilution from stock solutions dispensed from the dispense tips 210 mounted on the dispense head 130. The syringe 160 (shown in FIGS. 1-3) takes in aqueous stock solution of the test compound from storage unit, and discharge it into each test well 190. Serial dilution is a stepwise dilution of a substance in solution. Typically, the dilution factor at each step is constant resulting in a geometric progression of the concentration in a logarithmic fashion, which establishes a range of toxicant concentrations. Any dilution factor can be chosen such that a relevant dose response curve is obtained. The probes 220 mounted on the dispense head 130 are used to record testing conditions (i.e. oxygen, pH or conductivity levels) of resulted test solutions inside of test well 190.

In some embodiments, cell density of test organism is obtained by digital image recognition. Referring now to FIG. 3 and FIG. 4B (enlarged portion 300), each sample holder 180 carrying multiple test wells 190 is equipped with the rotational mechanism 331 which can move one test well 190 next to the electronic detector 150. A digital picture can be captured by an image sensor, such as charged coupled devices (CCD) or complementary metal oxide semiconductor detectors (CMOS). The cell density is obtained by applying analysis on the digital picture using imaging software installed on a computer connected with the disclosed apparatus. The dose response relationship of test organisms exposed to test compound is thereafter determined. If endpoints such as No Observable Effect Level (NOEL) and Inhibition Concentration 50% (IC50) are not established, further serial dilution of test compound may be warranted. The automated toxicity assessment apparatus 100 can include modules allowing a high degree of automation capable of assessing the toxicity of a high number of test compounds. The disclosed apparatus can reuse multiple test wells 190 on each motorized sample holder 180. Each test well 190 is separated from a drain channel 321 underneath by a semi-porous filter 310 for filtering the test solution to be drained out. At the end of each testing circle, used testing solution from each test well 190 can be discharged via a main drain channel 320 under the carousal system 140 into the attached waste storage by vacuum or pressurized clean air. The test wells 190 can be washed and cleaned through the dispense tip 210 comprises the syringe 160 which takes in reagent water (distilled or deionized water that does not contain substances which are toxic to the test organisms) from a storage unit. Thus, the disclosed apparatus can obtain toxicity data from chemical compounds at high throughput. The disclosed apparatus provides means for storing a sample solution comprising test organisms, means for serial dilution of chemical compound of which the toxicity is to be assessed, and means for continuous automatic measurement of dose-response relationship of test compound.

The control unit 170 can include a temperature control unit, RS232C serial port, p-COM, CAN BUS, and data acquisition card. In some embodiments, the disclosed apparatus can include a temperature control unit that employs Peltier device and heat radiation board for temperature control. A thermostatic system of the apparatus that ensures uniform temperature inside the enclosure 110, can minimize measuring error, which may be caused by temperature change, and also can secures long-term storage of microorganism. The control unit 170 can control the operations of the stepping motors that controls precise positions of the syringe 160 or the robotic arm 120, the analog-digital converter that converts the output data of the probes 220 into digital data that can be acknowledged by the computer, various solenoid valves that convert electric signal into mechanical signal, the power supplier that supplies required DC power, the temperature sensor that measures the interior temperature of the apparatus and/or the power controller that controls the heat generated from peltier device, the pH, oxygen level, specific conductivity sensors that monitor physical conditions of test solution comprising test organisms.

In some embodiments, a graphic user interface (GUI) commercialized for a personal computer is employed in the presently disclosed apparatus. Thus, all information regarding to the operation status of the apparatus can be processed through the monitor screen of the computer. Acquired data regarding to dose-response curves of test organisms can be stored and processed through a database system which is accessible remotely. In addition, through the control unit, self-diagnosis, and detection of exterior environment change and respond thereto are processed automatically to secure the safety of the apparatus. The disclosed apparatus can be controlled remotely and/or automatically operates to measures toxicity of test compounds, and processes the data obtained there for a prescribed period, without the operator's manipulation by using reagents and organisms stored in this apparatus.

Only a few examples and implementations are described. Other implementations, variations, modifications and enhancements to the described examples and implementations may be made without deviating from the spirit of the present invention. For example, the liquid transfer, metering, and dispense can be implemented using different mechanisms from the examples disclosed above. Moreover, the relative movements between the liquid dispense system, the test cells, and the electronic detector can be achieved by other means from the exemplified implementations described.

What is claimed is:

1. An automated toxicity assessment apparatus, comprising:
a plurality of sample plates each configured to hold multiple test wells each configured to hold a test solution comprising a test compound and a test organism, wherein the plurality of sample plates comprises a first sample plate;
a dispense head configured to hold a plurality of dispense tips configured to dispense test solutions into the test wells in one of the plurality of sample plates at each time;
an electronic detector configured to capture an image of a test solution in one of test wells in one of the plurality of sample plates, which allows a dose-response curve to be established for the test organism; and
a carousal system configured to rotate the first sample plate to a first position under the dispense head to allow the plurality of dispense tips to dispense solutions respectively into the test wells on the first sample plate, wherein the carousal system is configured to rotate the first sample plate to a second position to allow the electronic detector to capture an image of a test solution in the one of test wells on the first sample plate.

2. The automated toxicity assessment apparatus of claim 1, wherein the dispense head is configured to hold a plurality of probes configured to measure oxygen levels, pH values, or conductivities in the test solutions in the test wells on the first sample plate.

3. The automated toxicity assessment apparatus of claim 1, further comprising:
a robotic arm configured to lower the dispense head to engage the plurality of dispense tips respectively with the test wells on the first sample plate when the first sample plate is rotated to the first position by the carousal system.

4. The automated toxicity assessment apparatus of claim 3, wherein the dispense head is configured to hold a plurality of probes configured to conduct measurements in the test solutions in the test wells on the first sample plate, wherein the robotic arm configured to lower the dispense head to engage the plurality of probes respectively with the test wells on the first sample plate when the first sample plate is rotated to the first position by the carousal system.

5. The automated toxicity assessment apparatus of claim 1, further comprising:
a rotational mechanism configured to rotate the first sample plate when the first sample plate is moved to the second position the carousal system, thereby allowing different test wells to be moved to the vicinities of the electronic detector and images of the different associated test solutions to be captured by the electronic detector.

6. The automated toxicity assessment apparatus of claim 1, further comprising:
a liquid transfer mechanism configured to transfer the test solution to the plurality of dispense tips.

7. The automated toxicity assessment apparatus of claim 6, wherein the liquid transfer mechanism comprises a syringe configured to draw the test solution from a storage unit and pump the test solution towards the plurality of dispense tips.

8. The automated toxicity assessment apparatus of claim 1, further comprising:
a drain channel in the first sample plate and configured to drain test solutions from the test cells.

9. The automated toxicity assessment apparatus of claim 8, further comprising:
a filter in the first sample plate and configured to filter the test solutions to be drained from the test cells.

10. The automated toxicity assessment apparatus of claim 1, wherein the test organism includes green alga selenastrum capricornutum.
